(19) United States
(12) Patent Application Publication
(10) Pub. No.: US 2025/0331480 A1
(43) Pub. Date: Oct. 30, 2025

(54) METHODS FOR INCREASING SEED YIELD AND OIL SEED PRODUCTION

(71) Applicant: THE BOARD OF TRUSTEES OF THE UNIVERSITY OF ILLINOIS, URBANA, IL (US)

(72) Inventor: Ken N. PAIGE, Monticello, IL (US)

(21) Appl. No.: 19/217,006

(22) Filed: May 23, 2025

Related U.S. Application Data
(63) Continuation of application No. 18/650,973, filed on Apr. 30, 2024, now abandoned.

(51) Int. Cl. A01H 3/04; A01N 25/30; A01N 37/02; A01P 21/00

(52) U.S. Cl. CPC A01H 3/04; A01N 25/30; A01N 37/02; A01P 21/00

(57) ABSTRACT

Compositions and methods for increasing the quantity of plant-based oil produced per hectare in a given time period are provided, wherein the compositions include the use of one or more fatty acids that kill or inhibit the growth of cells in the shoot apical meristem and one or more surfactants.

### CROSS-REFERENCE TO RELATED APPLICATIONS

This application is a continuation of U.S. Ser. No. 18/650,973 filed Apr. 30, 2024, the content of which is incorporated herein by reference in its entirety.

### BACKGROUND

Plant-based oils are important for use in food, industrial products, and biofuels. Because of global warming, biofuels such as biodiesel will be important energy sources in the future and are receiving increased attention as a sustainable alternative to petrochemicals.

Biodiesel is a mono-alkyl ester of long chain fatty acids. Biodiesel can be produced from vegetable oil, animal oil/fat, tallow, and waste cooking oil. Transesterification is a suitable process to convert oils and fats into biodiesel. Transesterification may involve the reaction of an alcohol, such as ethanol, with the triglycerides present in oils, fats or recycled grease to form fatty acid alkyl esters (biodiesel) and glycerol. The reaction requires heat and a strong base catalyst such as sodium hydroxide or potassium hydroxide.

Biodiesel has better properties than petroleum-based diesel because it is renewable, non-toxic, exhibits excellent lubricity, is essentially free of sulfur and aromatics, and has higher biodegradability than fossil fuels. Biofuels can be used in any diesel engine without the need to redesign current technologies. In addition, biodiesel does not contribute to carbon dioxide levels in the atmosphere.

The most widely used feedstocks for biofuel production are vegetable oils or plant oils. Oilseed plants commonly used as feedstocks for biodiesel production include, e.g., soybean, rapeseed, canola, camelina, sorghum, safflower, sunflower, corn, sesame, cotton, cress, peanut, castor, flax, cress, mustard, quinoa, chia, cuphea, and amaranth. Oil obtained from the seeds of castor and rapeseed are not typically used as food and therefore their use in the manufacture of biofuels does not compete with food applications.

There is a limited amount of high-quality arable land for cultivation of plants for food, fiber, and biofuels. There is a tremendous need to significantly increase crop yields while at the same time limiting use of fertilizers and other agrochemicals (pesticides and herbicides) that add to the carbon footprint of agriculture.

### SUMMARY OF THE INVENTION

Provided herein is a method of increasing a quantity of seed oil produced per hectare in a 12-month time period by applying an effective amount of a composition comprising one or more fatty acids and one or more surfactants to the shoot apical meristem of a population of oilseed plants thereby increasing the quantity of seed oil produced per hectare in a 12-month time period by the population of oilseed plants as compared to a population of untreated control plants.

Also provided is a method of increasing seed yield of an oilseed plant produced per hectare in a 12-month time period comprising applying an effective amount of a composition comprising one or more fatty acids and one or more surfactants to the shoot apical meristem of a population of oilseed plants thereby increasing seed yield of the oilseed plant produced per hectare in a 12-month time period as compared to a population of untreated control plants.

### BRIEF DESCRIPTION OF THE DRAWINGS

FIG. 1 shows seed production adjusted for percent oil content in soybean, Glycine max, in an overcompensating soybean cultivar after removal of the shoot apical meristem at the V2/V3 stage of development. Plants in which the apical meristem was removed showed a 38.6% increase in the number of seeds produced adjusted by the average percent oil produced/seed. Hence, plants with the apical meristem removed produced significantly more oil seed than control plants.

### DETAILED DESCRIPTION OF THE INVENTION

The following descriptions and examples illustrate methods for increasing seed yield and oil obtained from oil seed crops. Although the present disclosure has been described in some details by way of illustration and example for purposes of clarity and understanding, it will be apparent that certain changes and modifications can be practiced within the scope of the appended claims.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment. It is to be understood that the present disclosure is not limited to the particular aspects described herein and as such can vary. Those of skill in the art will recognize that there can be variations and modifications of the present disclosure, which can be encompassed within its scope.

It is intended that every maximum numerical limitation given throughout this specification include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All terms are intended to be understood as they would be understood by a person skilled in the art. Unless defined otherwise, all technical and scientific terms used herein have the same meaning as commonly understood by one of ordinary skill in the art to which the disclosure pertains.

In this application, the use of the singular includes the plural unless specifically stated otherwise. It must be noted that, as used in the specification, the singular forms “a,” “an,” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean A individually; B individually; C individually; A and B; B and C; A and C; and A, B, and C. The term “or” can be used conjunctively or disjunctively unless the context specifically refers to a disjunctive use.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any aspect described herein can be implemented with respect to any method or composition herein, and vice versa. Furthermore, compositions of the present disclosure can be used to achieve the methods described herein.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed. The term “under suitable condition” or “under suitable reaction condition” refers to any environment that permits a desired reaction to take place.

The term “isolated” refers to a state where it is partially, substantially, or completely free of the materials with which it is associated in nature. By partially or substantially free is meant at least 0.1%, at least 0.5%, at least 1%, at least 5%, at least 10%, at least 20%, at least 30%, at least 40%, at least 50%, at least 60%, at least 70%, at least 80%, at least 90%, at least 99%, or 100% free of the materials with which it is associated in nature, inclusive of all values falling in between these percentages. Accordingly, as used herein, an “isolated fatty acid” refers to a fatty acid that has been partially, substantially, or completely separated from its biological source (e.g., microbial organism, yeast, bacteria, etc.). The isolated fatty acid may or may not be combined in a formulation with other ingredients for application disclosed herein. An isolated fatty acid may or may not be purified (e.g., free from other environmental contaminants, microbial secretes, or deactivated organisms, etc.), but it is separated from the source organisms.

The present invention is based, in part, on the discovery that fatty acids, when sprayed on or applied to a plant, selectively remove the shoot apical meristem, which results in an increase in the level of endopolyploidy leading to an increase in the quantity of seed yield and plant-based oil and hence seed oil production per hectare in a given time period, in particular a 12-month time period. Advantageously, application of the fatty acids inhibits the growth or kills a significant number of cells in the shoot apical meristem, without significantly damaging growth of other parts (e.g., stems, leaves, or roots) of the plant.

Accordingly, the invention provides methods of increasing seed yield and a quantity of seed oil of an oilseed plant produced per hectare in a 12-month time period by applying an effective amount of a composition comprising one or more fatty acids and one or more surfactants to the shoot apical meristem of a population of oilseed plants thereby increasing seed yield and a quantity of seed oil of the oilseed plant produced per hectare in a 12-month time period as compared to a population of untreated control plants (i.e., plants that have not been treated with a composition described herein). A population of plants is intended to refer to more than one plant and preferably includes a field, plot, acre, hectare (i.e., about 2.47 acres or 10000 square meters), or other unit of plants used in agricultural production.

As used herein, the term “seed yield” refers to a measurable amount of seed (grain) produced by a population of plants. “Seed yield” may be measured by one or more of the following parameters: number of seeds or number of filled seeds (per plant or per area (acre/square meter/or the like)); seed filling rate (ratio between number of filled seeds and total number of seeds); number of flowers per plant; seed biomass or total seed weight (per plant or per area (acre/square meter/or the like); thousand kernel weight (TKW; extrapolated from the number of filled seeds counted and their total weight; an increase in TKW may be caused by an increased seed size, an increased seed weight, an increased embryo size, and/or an increased endosperm). Seed yield may be determined on a dry weight or on a fresh weight basis, or typically on a moisture adjusted basis, e.g., at 15.5 percent moisture.

“Increased seed yield” means that the seed yield of the respective population of plants is increased by a measurable amount over the seed yield of the same population of plants produced under the same conditions, but without removing the shoot apical meristem of the plants. Increased seed yield may be characterized by, e.g., increased seed number and/or increased seed weight. In some aspects, improved or increased seed yield refers to enhanced yield of seeds in terms of either dry weight or fresh weight, or both. In one aspect, an increase in seed yield refers to increased harvestable seed yield. Seed yield in accordance with the methods herein may be calculated on a per plant basis or in relation to a specific area, seed yield per acre/square meter/or the like. In some aspects, seed yield may be calculated in relation to a hectare of land, i.e., seeds produced per hectare. Seed yield in accordance with the methods herein may also be calculated based upon a predetermined time period, e.g., months, years, or growing session. In some aspects, seed yield may be calculated based upon a 12-month time period.

“Seed oil” refers to the oil extracted or extractable from seeds of an oilseed plant. “Increased quantity of seed oil” means that the amount or quantity of seed oil obtained from the respective population of plants is increased by a measurable amount over the amount or quantity of seed oil obtained from the same population of plants produced under the same conditions, but without removing the shoot apical meristem of the plants. In one aspect, an increase in the quantity of seed oil refers to increased harvestable seed oil. The quantity of seed oil in accordance with the methods herein may be calculated on a per plant, per seed, or per 10 seed basis or in relation to a specific area, seed oil obtained per acre/square meter/or the like. Mass of oil per plant may also be used as a unit of measure and may be calculated from total seed weight. In some aspects, seed oil obtained from a population of plants may be calculated in relation to a hectare of land, i.e., seed oil produced per hectare. Seed oil in accordance with the methods herein may also be calculated based upon a predetermined time period, e.g., months, years, or growing session. In some aspects, seed oil produced may be calculated based upon a 12-month time period.

Seed oil content may be measured by conventional techniques. For example, Nuclear Magnetic Resonance Spectrometry (NMR) is used for determining oil, protein, and moisture content in seeds of various oilseed crops. The instrument is calibrated for several oil crops including soybean, camelina, canola, meadowfoam, and euphorbia, and can be calibrated for other species as needed. Tests can be performed in whole seeds or meals. The oil content test estimates the amount of oil that can be extracted from seeds using an industrial extraction method. Direct pressing extracts 90-92%, whereas solvent extractions remove 97-99% of oils contained in seeds. The NMR technique measures the resonance energy absorbed by hydrogen atoms in the sample. Usually oil contents are expressed based on a specific moisture basis (e.g., 8.5%, 10%, etc.). Using the NMR in measuring oil content is referenced by ISO 10565:1998 Oilseeds. The typical oil content in seeds of different feedstock can vary, e.g., oil content of castor seeds, soybean, sunflower seeds, and rapeseed is 40-55%, 15-20%, 25-35%, and 38-46%, respectively.

The “harvestable yield” of a plant can depend on the specific plant/crop of interest as well as its intended application (such as food production, feed production, processed food production, biofuel, biogas or alcohol production, or the like) of interest in each particular case. Thus, in one aspect, yield is calculated as harvest index (expressed as a ratio of the weight of the respective harvestable parts (e.g., seed or seed oil) divided by the total biomass), harvestable parts weight per area (acre, square meter, or the like), and the like.

In one aspect, seed yield and/or quantity of seed oil may be increased by at least 5% to 45% (or any range derivable therein) compared to untreated controls. In one aspect, seed yield and/or quantity of seed oil are increased by least 10% compared to untreated controls. In one aspect, seed yield and/or quantity of seed oil may be increased by at least 20% compared to untreated controls. In one aspect, seed yield and/or quantity of seed oil may be increased by at least 30% compared to untreated controls. In one aspect, seed yield and/or quantity of seed oil may be increased by at least 35% compared to untreated controls. In one aspect, seed yield and/or quantity of seed oil may be increased by at least 40% compared to untreated controls. In one aspect, seed yield and/or quantity of seed oil may be increased by at least 45% compared to untreated controls. By way of example, if untreated soybeans yield 6200 bushels of seeds per 40 hectares, and if soybeans that received the subject treatment yield 8500 bushels of seeds per 40 hectares under the same growing conditions, then the yield of soybeans would be said to have increased by ((8500−6200)/6200)×100=37%. In some aspects, per hectare yield or and/or quantity of seed oil can be increased by 1%, 5%, 10%, 15%, 20%, 25%, 30%, 35%, 40%, 45%, 50%, 60%, 70%, or more in a 12-month time period through the removal, or inhibition of growth, of the plant apical meristem.

In another aspect, when two oilseed crops (crop 1 and crop 2) are treated to remove, or inhibit the growth of, the shoot apical meristem, the seed yield and/or seed oil yield produced by oilseed crop 1 and/or oilseed crop 2 in a 12-month period on a defined amount of land is increased compared to untreated control crops 1 and 2. The increase in seed yield or quantity of seed oil is the combined increase in yield for crop 1 and crop 2 and may be 1%, 5%, 10%, 15%, 20%, 25%, 30%, 35%, 40%, 45%, 50%, 55%, 60%, 65%, 70%, 75%, 80%, 85%, 90%, 95%, 100%, or more than 100%, compared to the seed yield or quantity of seed oil obtained from untreated oilseed crop 1 and crop 2. The oilseed crop 1 and crop 2 can be any of soybean, rapeseed, canola, safflower, sunflower, sesame, cotton, cress, castor bean, or another oilseed crop.

In some aspects, the increase in seed yield in a population of plants contacted with the composition described herein is comparable or better than the improvement in seed yield of a population of plant subjected to clipping to remove the apical meristem. The term “clipping” means removal or inhibition of the growth of the shoot apical meristem of a plant by any means of mechanical trimming. Mechanical trimming is accomplished by mowing, pruning by hand, or any other method of severing the apical meristem in whole, or in part, from the plant.

The term “plant” is to be understood as a plant of economic importance and/or cultivated plant. A plant is preferably selected from an agricultural, silvicultural and horticultural (including ornamental) plant. The term “plant” as used herein includes all parts of a plant such as germinating seeds, emerging seedlings, herbaceous vegetation as well as established woody plants including all belowground portions (such as the roots) and aboveground portions. Generally, the term “plant” also includes a plant that has been modified by breeding, mutagenesis or genetic engineering. Genetically modified plants are plants, which genetic material has been modified by the use of recombinant DNA techniques. The use of recombinant DNA techniques makes modifications possible that cannot readily be obtained by cross breeding under natural circumstances, mutations or natural recombination.

In some aspects, a plant of the invention is an oilseed plant. An “oilseed plant” is a plant that is grown or cultivated for production of oils from the seeds of the plant, e.g., edible oils, edible oils used as food supplements or nutraceutical, multipurpose oils, inedible oils as biofuel, drying oils, and/or oils used in industrial applications and commercial products. In some aspects, the oilseed plant is a plant grown or cultivated as a row crop. Edible oils include the following, which are also used as fuel oils, corn oil, cottonseed oil, peanut oil, rapeseed oil, safflower oil, sesame oil, soybean oil, and sunflower oil. Melon and gourd seed oils are obtained from members of the Cucurbitaceae, including gourds, melons, pumpkins, and squashes. Examples of such oils include bitter gourd oil from seeds of Momordica charantia, bottle gourd oil from seeds of Lagenaria siceraria, buffalo gourd oil from seeds of Cucurbita foetidissima, butternut squash seed oil from seeds of Cucurbita moschata, Egus seed oil from seeds of Cucumeropsis mannii naudin, pumpkin seed oil, and watermelon seed oil from seeds of Citrullus vulgaris.

Other edible oils include Amaranth oil from the seeds of grain amaranth species, including Amaranthus cruentus and Amaranthus hypochondriacus; false flax oil from seeds of Camelina sativa; hemp oil; meadowfoam seed oil; mustard oil; okra seed oil from Abelmoschus esculentus; Quinoa oil; ramtil oil pressed from the seeds of the one of several species of genus Guizotia abyssinica (Niger pea); rice bran oil; thistle oil pressed from the seeds of Silybum marianum; tobacco seed oil from the seeds of Nicotiana tabacum and other Nicotiana species; tomato seed oil; and wheat germ oil.

Oils used as food supplements or nutraceuticals include black seed oil pressed from Nigella sativa seeds, borage seed oil from the seeds of Borago officinalis, evening primrose oil from the seeds of Oenothera biennis, and flaxseed oil (called linseed oil when used as a drying oil) from the seeds of Linum usitatissimum.

Oils used primarily for human consumption, but which have been considered for use as biofuels, i.e., multipurpose oils, include castor oil; colza oil from Brassica rapa var. oleifera (oilseed turnip); corn oil; cottonseed oil; false flax oil from Camelina sativa; hemp oil; mustard oil; peanut oil; radish oil; rapeseed oil; rice bran oil; safflower oil; soybean oil; and sunflower oil.

Vegetable oils that dry to a hard finish at normal room temperature are referred to as “drying oils” and are used as the basis of oil paints and in other paint and wood finishing applications. Such oils include sunflower and safflower oil; linseed (flaxseed) oil; and vernonia oil produced from the seeds of the Vernonia galamensis.

Other plant oils of importance that are either inedible, or which are not commonly ingested as edible oils, can be used for a wide variety of other purposes including, for example, insecticides, perfumes, various industrial applications, sources of triglycerides and fatty acids, medicinal and cosmetic uses, etc. These include, for example, bladderpod oil pressed n the seeds of Lesquerella fendleri; carrot seed oil pressed from carrot seeds; castor oil; crambe oil extracted from seeds of Crambe abyssinica; and honesty oil from seeds of Lunaria annua.

In some aspects, the oilseed plants used in the methods described herein are soybean, rapeseed, canola, camelina, sorghum, safflower, sunflower, corn, sesame, cotton, cress, peanut, castor, flax, cress, mustard, quinoa, chia, cuphea, or amaranth plants.

Soybean (Glycine max) oil is a co-product with soybean meal. To manufacture biodiesel from soybean oil, harvest beans are crushed, and the meal and oil separated.

Rapeseed (Brassica napus subsp napus) has a deep root system that allows the plants to scavenge for water and nutrients. The rapeseed plant is allowed to grow and mature normally until bolting, when the main flower stem begins to develop. When the main flower stem is still young, the apical meristem is removed which forces the plant to branch from remaining nodes. Low erucic acid varieties of canola also may be used. The plants are allowed to grow and mature normally until bolting, when the main flower stem begins to develop. When the main flower stem is still young, it is removed thereby removing apical dominance and forcing the plant to branch from remaining nodes.

Mustard plants, including black mustard (Brassica nigra), brown mustard (B. juncea), and white mustard (Sinapis alba), are similar to canola and rapeseed. Mustard is an excellent cover crop with excellent potential as a biodiesel feedstock. Although mustard produces less oil than rapeseed or canola, it is more drought tolerant and grows well on marginal soils. The mustard plant is allowed to grow and mature normally until bolting, when the main flower stem begins to develop. When the main flower stem is still young, it removed thereby removing apical dominance and forcing the plant to branch from remaining nodes.

Camelina (Camelina sativa) is an oil seed crop that is a relative of mustard, rapeseed, and canola. In some locations, camelina can be grown at a lower cost than rapeseed or canola because it requires less fertilizer and pesticides. Camelina has many favorable attributes for biodiesel production including high plant yield, high oil content in the seed, high net energy ratio, and relatively low production cost. Camelina has many favorable attributes for fuel production including high plant yield, high oil content in the seed, high net energy ratio, and comparatively low production cost. Camelina has a short life cycle of about 85-100 days from seeding to maturity making it an attractive crop for oil seed production.

Safflower (Carthamus tinctorius) is a highly branched, thistle-like annual in the family Asteracease in which vegetable oil is extracted from seeds. Safflower originates from a leafy rosette from which a branched central stem emerges. The main shoot reaches heights of 12-59 inches. Lateral branches develop once the main stem is about 7.9-15.7 inches high. These lateral branches can then branch again to produce secondary and tertiary branches. The chosen variety as well as growing conditions influence the extent of branching. The primary meristem can be removed anywhere from growth state 1 to growth stage 6 or higher on safflower during its growth period prior to flowering to enforce branching and the production of additional fruits.

Flax (Linum usitatissimum) is an herbaceous annual in the family Linaceae that is cultivated for its fiber, which produces linen, and for its seed, called flaxseed or linseed, from which linseed oil is produced. It has the highest alpha-linolenic acid levels of any other vegetable oils. Flax seeds are generally made up of approximately 20% protein and 41% oil concentrations. When flax germinates, the first parts to emerge from the ground are the cotyledons. These expand and from the middle of them, the apical meristem grows upwards, putting out new leaves. Three pairs of opposite, true leaves are the next to grow. The stem then continues to elongate, putting out small leaves that spiral up the main stem, until branching begins. All of this is part of the vegetative stage of the plant, which takes about 45-60 days. The flowering period (the reproductive stage) takes 15-25 days. The flowering period is when branching begins near the top of the plant and flowers emerge. Flowers remain open for approximately a day, and then close up while flax seeds mature inside the pod. Seeds mature and the plants dry down, which takes another 30-40 days.

Removal of the apical meristem can occur just above the first set of true leaves, the second set of true leaves, or the third set of true leaves, early in the life cycle of the plant. This is achieved, e.g., by treating the apical meristem with a chemical composition that kills sufficient cells, or inhibits the growth of sufficient cells, to stop or inhibit the growth of the apical meristem. This results in a few large branches low on the plant. How many true leaves are remaining determines how many potential branches can be formed.

Sesame (Sesamum indicum) is a perennial plant and is one of the oldest domesticated oilseed crops. It tolerates drought conditions well and has high oil concentrations in the seeds, generally about 60%. Sesame grows 20-39 inches tall, with opposite leaves 2 to 6 inches long, lanceolate in shape, 2 inches broad at the base of the plant, narrowing to just 0.4 inches broad on the flowering stem. Fruit capsules containing 4-12 seeds are produced from the base of the leaf nodes. The primary meristem can be removed anywhere from growth stage 1 to the growth stage 6 or higher on sesame during its growth period prior to flowering to enforce branching and the production of additional fruits.

Sunflower is a large annual in the genus Helianthus. Wild H. annuus is widely branched with many flower heads. The domestic sunflower often possesses only a single large inflorescence on an unbranched stem. Oil is extracted from sunflower seeds. After emergence of a sunflower plant from seed, the cotyledons expand with opposite nodes, followed by the first set of true leaves, also from opposite nodes. The stem elongates adding leaves alternating up the plant until the flower(s) develop. After pollination, each of the flowers produce seeds, which then develop, mature, and dry. Removal of the apical meristem can be achieved by applying a composition therein at the main stem just above the cotyledonary nodes and below the next node of opposite leaves. Apical meristem removal at this time leaves 2 remaining nodes from which the plant can grow branches. Apical meristem removal can also take place before the next higher leaves are fully formed or after they have formed. Apical meristem removal can also take place above the set of opposite nodes (which are just above the cotyledonary nodes). This can also occur before the leaves of the next node fully form or after they form. Apical meristem removal at this stage leaves 4 potential nodes from which branches can form. Most commonly, for both of these heights, 2 branches form from opposite nodes (either cotyledonary or the set higher) and both act like main stems. Alternatively, the apical meristem of the sunflower plant can be removed above one of the next approximately 5 leaves that are alternately located up the main stem. This can result in 1-3 branches forming from the highest remaining nodes.

The term “meristem” means a region of cells capable of division and growth in plants. Meristematic cells are typically small and nearly spherical. They have a dense cytoplasm and relatively few small vacuoles. Some of these meristematic cells maintain the meristem as a continuing source of new cells and may undergo cell division (mitosis) many times before differentiating into specific cells required for that region of the plant body.

As is conventional in the art, the “shoot apical meristem” is the region in the growing shoot containing meristematic cells. The shoot apical meristem contains multipotent stem cells and produces primordia that develop into all the above ground organs of a plant including the floral meristems. The plant hormone auxin is produced in the shoot apical meristem. Among the many roles of auxin in plant development, it inhibits the production of lateral branches.

The term “lateral meristems” means the meristem in the vascular and cork cambia. Lateral meristems are known as secondary meristems because they are responsible for secondary growth or increase in stem girth and thickness. The term “intercalary meristem” means the meristem at the internodes or stem regions between the places at which leaves attach.

In accordance with the methods herein, an effective amount of a composition comprising one or more fatty acids and one or more surfactants is applied to the shoot apical meristem of a population of oilseed plants. As used herein, the term “applying,” “applied,” “application,” “contact,” “contacting” or variations thereof, with reference to the composition means that the shoot apical meristem is contacted with the composition using any suitable means, e.g., sprayed, drenched, or misted. Various applicators may be used to apply the composition to the shoot apical meristem including, e.g., a hand-held aspirator-type sprayer or other commercial sprayer. Examples of commercial sprayers include the Hagie STS12, STS16 and STS20 models capable of carrying 1200, 1600 and 2000 gallons, respectively, of spray product. Such commercial sprayers may need to be modified with spray arms to target the apical meristems from the upper side of the plants. In some aspects, the spray includes a pressurized nozzle, e.g., a nozzle having a psi of about 40.

To effect overcompensation or an increase in the level of endopolyploidy leading to an increase in seed yield and/or the quantity of seed oil, some aspects provide for the killing or inhibition of the growth of the shoot apical meristem at a specified growth stage. The growth stage at which a composition herein is applied to the shoot apical meristem may be dependent upon the oilseed plant species/variety, geographical location, desired increase in seed yield and/or oil production, as well as other factors including environmental conditions such as soil, temperature, light, disease pressure, water availability, drought, nutrient availability, which are known to those of skill in the art to influence plant growth.

Depending, in part, on genetics, a plant selection, plant variety, inbred plant, or hybrid plant may not express overcompensation when cells in the apical meristem are killed or their growth inhibited. Thus, plants may be screened to identify those capable of expressing overcompensation, e.g., by treatment of the shoot apical meristem with a composition described herein or clipping and measuring seed yield or quantity of seed oil to determine whether there is an increase in seed yield or quantity of seed oil in the treated plant as compared to a plant not treated with the composition or clipped. Furthermore, the optimal time in the plant growth cycle for removal, or inhibition of growth, of the apical meristem may be determined by treatment of the shoot apical meristem with a composition described herein at different stages of growth and determining the stage in which there is an increase in seed yield or quantity of seed oil as compared to a plant not treated with the composition.

Alternatively, or in addition to, screening of plants and/or growth stage may be conducted at the molecular level. Increasing chromosome number through endoreduplication and therefore gene copy number provides a means of increasing expression of vital genes or genetic pathways that promote rapid regrowth rates following removal, or inhibition of growth, of the apical meristem. Glucose-6-phosphate dehydrogenase (G6PD1) feeds compounds into the oxidative pentose phosphate pathway for nucleotide biosynthesis, by the provision of ribose-5-phosphate, necessary for the significant increase in chromosome number via endoreduplication. The increase in DNA content then feeds back positively on pathways involved in metabolism (e.g., G6PD1) and chemical defense. Endoreduplication leads to increased gene copy number and therefore increased gene expression. Accordingly, gene expression of one or more genes of these pathways may be measured to assess appropriate plants and/or growth stages for use in this invention.

It is understood that a skilled person will be able to determine/identify the different growth stages of a particular crop concerned based upon conventional industry scales. Particular guidance for determining the growth stage of soybean (Glycine max L.) is provided by Soybean Growth Stages, published by the University of Illinois in 1999 (see, e.g., Najafikhan-Behbin, et al. (2019) Appl. Ecol. Environ. Res. 17(2):2911-2929) and McWilliam, et al. (1999) Soybean Growth and Management Quick Guide, A-1174, North Dakota State University, Frago, ND. Guidance for determining the growth stages of camelina (Camelina sativa) may be found in Martinelli & Glasso (2011) Annals Appl. Biol. 158:87-94. Guidance for determining the growth stages of peanut (Arachis hypogaea L.), cotton (Gossypium hirsutum L.), corn (Zea mays L.), canola (Brassica napus L.), and sunflower (Helianthus annuus L.) may be found in Meier (2001) Biologische Bundesanstalt, Bundessortenamt and Chemical (BBCH) Monograph. The BBCH scale uses a decimal code system, which is divided into principal and secondary growth stages, and is based on the cereal code system (Zadoks scale) developed by Zadoks et al. (1974) Weed Res. 14:415-421. Guidance for determining the growth stages of sorghum may be found at the Sorghum Checkoff Industry Organization Website.

By way of illustration, removal, killing of a significant number of cells, or inhibition to growth of the shoot apical meristem of soybean (Glycine max L.) may occur between (i) growth stage 1 and growth stage 2, (ii) growth stage 2 and growth stage 3, (iii) growth stage 3 and growth stage 4, (iv) growth stage 4 and growth stage 5, or (v) growth stage 5 and growth stage 6. For soybeans, VE is the stage at which cotyledons emerge and supply energy for the plant for 7 to 10 days. VC is the stage soon after the cotyledons are fully exposed. At VC unifoliate leaves emerge at the second node and begin generating energy through photosynthesis. Growth state 1 or the V1 stage is when the first trifoliate leaflets form. The V2 stage (growth stage 2) is when the second trifoliate leaf is established. At V2 root nodules begin to develop. Nitrogen fixation by the plant begins to occur when the plants reach 6 to 8 inches in height. V3 (growth stage 3) is the stage when the third trifoliate leaf emerges. V4 (growth stage 4) is when the fourth trifoliate leaf emerges. V5 (growth stage 5) is when the fifth trifoliate leaf emerges. The V6 stage (growth stage 6) is when plants develop new growth stages about every 3 days depending on the environment. At this stage lateral roots should overlap in 30-inch rows or less. At R1 flowering begins on the third to sixth node and continues up and down the main stem. Flowing eventually moves to the branches. Nodes on the main stem usually have at least one flower. Vertical roots as well as secondary roots and root hairs continue to grow rapidly until R4 and R5. At stage R2 an open flower develops at one of the top two modes of the main stem. The plant has accumulated about 25% of its total dry weight and nutrients and about 50% of its mature height. Nitrogen fixation by root nodules increases rapidly at this stage. At stage R3 a pod on at least one of the upper four nodes is at least 3/16-inch long or longer. At stage R4 the pods are growing rapidly, and seeds are developing. At stage R5 at least one ⅛-inch-long seed is present in a pod at one of the four upper-most nodes. About half of the nutrients required for seed filling come from the plant's vegetative parts and about half from Nitrogen fixation and nutrient uptake by the roots. Nitrogen fixation peaks at this stage. Stage R6 is known as the “green bean” stage because it marks the beginning of the full seed stage. At stage R7 at least one normal pod on the main stem reaches its brown or tan mature color. Seed dry matter begins to peak. Seeds and pods begin to lose green color. At stage R8 at least 95% of the pods on a plant have reached their mature color and the plant is fully mature. Typically, about 5 to 10 days of good drying weather after stage R8 is needed to obtain a harvest seed moisture content of less than 15%.

For corn (Zea mays), a composition as described herein may be applied to the shoot apical meristem of the corn between (i) growth stages 9 and 10, (ii) growth stages 10 and 11, (iii) growth stages 11 and 12, (iv) growth stages 12 and 13, (v) growth stages 13 and 14, (vi) growth stages 14 and 15, (vii) growth stages 15 and 16, (viii) growth stages 16 and 17, (ix) growth stages 17 and 18, (x) growth stages 18 and 19, or (xi) growth stages 19 and 30. Similar to dicots, growth stage of a monocot such as corn is when the first round-tipped leaf on first collar appears, and nodal roots elongate. By growth stage 2, the monocot may be 2 to 4 inches tall and rely on energy in the seed. Growth stage 3 begins 2 to 4 weeks after VE (emergence), and the plant switches from kernel reserves to photosynthesis and nodal roots begin to take over. Notably, in corn, a plant with 3 collars is considered growth stage 3, however, there may be 5 to 6 leaves showing on the plant. At the growth stage 4 stage, the fourth leaf collar is visible. At growth state 5-6, leaf collars are visible, the growing point is above the soil surface, the critical period of nitrogen uptake begins, and kernel row numbers are determined. See the Biologische Bundesanstalt, Bundessortenamt and Chemical (BBCH) scale for corn.

In some aspects, the oilseed plant is canola and the composition is applied to the shoot apical meristem of the canola between (i) growth stages 10 and 11, (ii) growth stages 11 and 12, (iii) growth stages 12 and 13, (iv) growth stages 13 and 14, (v) growth stages 14 and 15, (vi) growth stages 15 and 16, (vii) growth stages 16 and 17, (viii) growth stages 18 and 19, or (ix) growth stages 19 and 20. See the BBCH scale for canola.

In some aspects, the oilseed plant is camelina and the composition is applied to the shoot apical meristem of the camelina between (i) growth stages 10 and 11, (ii) growth stages 11 and 12, (iii) growth stages 12 and 13, (iv) growth stages 13 and 14, (v) growth stages 14 and 15, (vi) growth stages 15 and 16, (vii) growth stages 16 and 17, (viii) growth stages 18 and 19, or (ix) growth stages 19 and 21. See the BBCH scale for camelina.

In some aspects, the oilseed plant is sunflower and the composition is applied to the shoot apical meristem of the sunflower between (i) growth stages 10 and 12, (ii) growth stages 12 and 14, (iii) growth stages 14 and 16, (iv) growth stages 16 and 18, or (v) growth stages 19 and 30. See the BBCH scale for sunflower. See the BBCH scale for sunflower.

In some aspects, the oilseed plant is cotton and the composition is applied to the shoot apical meristem of the cotton between (i) growth stages 10 and 11, (ii) growth stages 11 and 12, (iii) growth stages 12 and 13, (iv) growth stages 13 and 14, (v) growth stages 14 and 15, (vi) growth stages 15 and 16, (vii) growth stages 16 and 17, (viii) growth stages 18 and 19, or (ix) growth stages 19 and 21. See the BBCH scale for cotton.

In some aspects, the oilseed plant is peanut and the composition is applied to the shoot apical meristem of the peanut between (i) growth stages 10 and 11, (ii) growth stages 11 and 12, (iii) growth stages 12 and 13, (iv) growth stages 13 and 14, (v) growth stages 14 and 15, (vi) growth stages 15 and 16, (vii) growth stages 16 and 17, (viii) growth stages 18 and 19, or (ix) growth stages 19 and 21. See the BBCH scale for peanut.

In some aspects, the oilseed plant is sorghum and the composition is applied to the shoot apical meristem of the sorghum between (i) growth stages 0 and 1, (ii) growth stages 1 and 2, (iii) growth stages 2 and 3, (iv) growth stages 3 and 4, (v) growth stages 4 and 5, or (vi) growth stages 5 and 6. See the Sorghum Checkoff Industry Organization developmental stages of the sorghum plant.

Ideally, removal of the shoot apical meristem results in minimal or no removal of adjacent growth stage. For example, the methods provide for minimal or no removal of adjacent growth stage 1 if removing the apical meristem between growth stage 1 and growth stage 2, or minimal or no removal of adjacent growth stage 2 tissue if removing the apical meristem between growth stage 2 and growth stage 3, or minimal or no removal of adjacent growth stage 3 tissue if removing the apical meristem between growth stage 3 and growth stage 4, or minimal or no removal of adjacent growth stage 4 tissue if removing the apical meristem between growth stage 4 and growth stage 5, or minimal or no removal of adjacent growth stage 5 tissue if removing the apical meristem between growth stage 5 and growth stage 6, etc.

Given that growth stages can overlap, a crop of plants is in a particular growth stage when 50% or more of the plants of the crop are in or beyond that stage. Moreover, if senescence of the lower leaves has occurred, leaf scars (excluding those where the cotyledons were attached) are counted to determine the proper stage. Removal or inhibition of the growth of the apical meristem may occur within the 3-to-10-day window between stages, e.g., between stages 1 and 2, stages 2 and 3, stages 3 and 4, etc. In addition, depending on the plant variety, geographic region, or agronomic conditions, the apical meristems of the plants may be removed early or later in plant growth.

Subsequent to treatment of the plant with the composition of this invention, the plant is grown for a sufficient amount of time to exhibit an increase in seed yield and seed oil quantity. In some aspects, the plant is grown to a stage when the seeds of the plant are harvestable. Accordingly, in some aspects, the methods herein further include the step of harvesting the seeds of the oilseed plant.

In some aspects, the composition used in the methods described herein includes one or more fatty acids (e.g., 1, 2, 3, 4 or 5 fatty acids) and one or more surfactants. In some aspects, the one or more fatty acids have the structure of formula (I):

X—C(O)—OR1 (I),

wherein X is a linear or branched C4-C30-alkyl group optionally containing 1 to 6 carbon-carbon double bonds, and R1 is H or C1-C6-alkyl. Representative examples of fatty acids to include in the composition for inhibiting the growth of the apical meristem include the following saturated fatty acids: caprylic (CH₃(CH)₈COOH), acid capric acid (CH₃(CH)₁₀COOH), lauric acid (CH₃(CH₂)₁₂COOH), myristic acid (CH₃(CH₂)₁₄COOH), palmitic acid (CH₃(CH₂)₁₆COOH), stearic acid (CH₃(CH₂)₁₈COOH), arahidic acid (CH₃(CH₂)₂₀COOH), behenic acid (CH₃(CH₂)₂₂COOH), lignoceric acid (CH₃(CH₂)₂₄COOH), and cerotic acid (CH₃(CH₂)₂₆COOH). Representative examples of fatty acids to include in the composition for killing or inhibiting the growth of the apical meristem include the following unsaturated fatty acids: myristoleioc acid (CH₃(CH₂)₃CH═CH(CH₂)₇COOH), palmitoleic acid (CH₃(CH₂)₅CH═CH(CH₂)₇COOH), sapienic acid (CH₃(CH₂)₈CH═CH(CH₂)₄COOH), oleic acid (CH₃(CH₂)₇CH═CH(CH₂)₇COOH), vaccenic acid (CH₃(CH₂)₅CH═CH(CH₂)₉COOH), and linoleic acid ((CH₃(CH₂)₄CH═CHCH₂CH═CH(CH₂)₇COOH).

In some aspects, the one or more fatty acids include methyl decanoate, 1-decanol, and/or methyl nonanoate. In some aspects, the fatty acid is methyl decanoate. In some aspects, the fatty acid is 1-decanol. In some aspects, the fatty acid is methyl nonanoate. In some aspects, the fatty acid is a combination of methyl decanoate and 1-decanol. In some aspects, the fatty acid is a combination of methyl decanoate and methyl nonanoate. In some aspects, the fatty acid is a combination of 1-decanol and methyl nonanoate.

In some aspects, the one or more fatty acids of the composition herein include a combination of lower alkyl esters of the C6 to C12 fatty acids and C6 to C12 fatty acid alcohols in aqueous emulsions. In some aspects, the one or more fatty acids include a combination of methyl esters of C6, C8, C10, and C12 fatty acids. In particular, the one or more fatty acids of the composition herein include a combination of: a methyl ester of a C6 fatty acid (methyl hexanoate), a methyl ester of a C8 fatty acid (methyl octanoate), a methyl ester of a C10 fatty acid (methyl decanoate), and a methyl ester of a C12 fatty acid (methyl laureate). In particular aspects, the composition is composed of about 4% methyl hexanoate; about 56% methyl octanoate; about 38% methyl decanoate; and about 2% methyl laureate, based on total amount of fatty acid of the composition.

In accordance with the methods described herein, an effective amount of a composition including one or more fatty acids and one or more surfactants is used. The effective amount used may be dependent upon the plant and/or plant variety being treated, the formulation of the composition, the mode or rate of application, environmental conditions, and/or growth stage in which the composition is applied. In general, the concentration of the one or more fatty acids in the composition may be at least about 0.01 M (e.g., 0.01, 0.015, 0.025, 0.03, 0.035, 0.04, 0.045, 0.05, 0.055, 0.06, 0.07, 0.08, 0.09, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0 M or more). In some aspects, the one or more fatty acids in the composition may be at least about 0.025 M. In some aspects, the one or more fatty acids in the composition may be at least about 0.05 M. In some aspects, the one or more fatty acids in the composition may be at least about 0.1 M. In some aspects, the one or more fatty acids are present in the composition at a concentration in the range of 0.025 M to 0.50 M (e.g., 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4 or 0.5 M). In some aspects, the one or more fatty acids are present in the composition at a concentration in the range of 0.025 M to 0.10 M.

In some aspects, the fatty acid component of the composition is in the range of about 20% to 60% (e.g., about 20%, 25%, 30%, 35%, 40%, 45%, 50%, 55%, or 60%) by weight of the composition. In some aspects, the fatty acid component of the composition is in the range of about 45% by weight of the composition. By way of illustration, the total quantity of the methyl hexanoate, methyl octanoate, methyl decanoate and methyl laureate in the composition may be about 3.72 lbs per gallon and each fatty acid is present at about 4%, 56%, 38%, and 2%, respectively, of total fatty acids.

In addition to one or more fatty acids, the composition herein also includes one or more surfactants (e.g., 1, 2, 3, 4, or 5 surfactants). As used herein, the term “surfactant” or “emulsifier” describes a chemical substance that acts as a stabilizer for emulsions, preventing liquids that are immiscible with one another from separating, typically by increasing the kinetic stability of the emulsion by, e.g., lowering the interfacial tension between the liquids. In some aspects, the surfactant is an inert component of the composition.

Surfactants typically have a lipophilic group and a hydrophilic group, and can be categorized as ionic (e.g., cationic, anionic or zwitterionic) or nonionic agents. In some aspects, the one or more surfactants are nonionic. In some aspects, the one or more nonionic surfactants include at least one fatty acid group, at least one polyethoxy group, or more than one ethoxy group, and at least one polyol (diol, triol or higher alcohol), wherein the fatty acid and polyol may be combined in hydroxyl-fatty acid. In some aspects, the one or more surfactants are polyoxyethylene sorbitan monoesters, such as the laurates, palmitates, stearates, and oleates. The preferred average number of oxyethylene groups per molecule is about 20, although sorbitan monoesters containing from about 10 to about 30 oxyethylene groups per molecule are similarly useful. In some aspects, the one or more fatty acids include one or more polyoxyethylene sorbitan esters, polyoxyethylene alcohols, alkylarylpolyether alcohols, phthalic glycerol alkyl resins, ethoxylated imidazoline, decanoic acid, diglycol oleate, polyether sulfonates, alkylaryl sulfonates, and mixtures thereof.

Representative examples of nonionic surfactants that are usable in the context of the methods herein include, without limitation, polyoxyethlene(20) sorbitan monolaurate (sold under the tradename TWEEN® 20), polyoxyethylene (4) sorbitan monolaurate (sold under the tradename TWEEN® 21), polyoxyethylene (2) sorbitan monopalmiate (sold under the tradename TWEEN® 40), polyoxyethlene (20) sorbitan monstearate (sold under the tradename TWEEN® 60), polyoxyethylene (20) sorbitan monooleate (sold under the tradename TWEEN® 80), ethoxylated imidazoline (ACL-429), polyoxyethylene fatty glyceride (G 1288 or G 1300) (polyoxyethylene fatty glyceride), and polyoxyethylene (12) dodecylophenol (Tergitoal 12-P-12).

In some aspects, the composition comprises one or more surfactants and one or more fatty acids comprising (i) methyl decanoate, 1-decanol, or methyl nonanoate; or (ii) a combination of methyl esters of C6, C8, C10, and C12 fatty acids. In some aspects, the composition consists of one or more surfactants and one or more fatty acids selected from the group of (i) methyl decanoate, 1-decanol, and methyl nonanoate; and (ii) a combination of methyl esters of C6, C8, C10, and C12 fatty acids. As used herein, “consists of” or “consisting of” is not meant to exclude a solvent (e.g. water or combination of inert solvents) into which the fatty acid and optional surfactant are dissolved/suspended. In some aspects, the composition comprises or consists of one or more surfactants and methyl decanoate. In some aspects, the composition comprises or consists of one or more surfactants about 4% of the methyl ester of the C6 fatty acid; about 56% of the methyl ester of the C8 fatty acid; about 38% of the methyl ester of the C10 fatty acid; and about 2% of the methyl ester of the C12 fatty acid, based on total fatty acid of the composition. In one aspect, the composition comprises or consists of methyl decanoate and polyoxyethylene sorbitan monolaurate. In some aspects, more than one surfactant is included in the composition. In some aspects, each of the surfactants (e.g., two or more surfactants) is present at an equal concentration by weight of the composition. In some aspect, the total amount of the one or more surfactants is equal in weight to the total weight of the one or more fatty acids. In other aspects, the one or more surfactants are present at a 1:1 ratio (or 1:2 or 2:1), by weight, with the one or more fatty acids of the composition.

In addition to one or more fatty acids, the composition of the invention may include one or more suitable fungicides, bactericides, and/or pesticides. The selection of the appropriate fungicide(s), bactericide(s), and/or pesticide(s) may be readily and empirically determined by methods well known to the skilled artisan and will vary depending on the plant species, geographic region, environmental factors, and other factors.

Suitable fungicides include, but are not limited to, Chlorothalonil, copper-based fungicides (such as Bordeaux mixture, copper hydroxide, copper sulfate), Mancozeb, Azoxystrobin, Pyraclostrobin, Bacillus subtilis, Trifloxystrobin, Tebuconazol, Propiconazole, Myclobutanil, Cyprodinil, Fenhexamid, thiophanate-methyl, Captan, and Difenoconazole.

Suitable bactericides include, but are not limited to, copper-based bactericides (such as Bordeaux mixture, copper hydroxide, copper sulfate), Streptomycin sulfate, Oxytetracycline (e.g., sold under the tradename MYCOSHIELD®), Bacillus subtilis, Bacillus amyloliquefaciens, Pseudomonas fluorescens, hydrogen dioxide (hydrogen peroxide), peroxyacetic acid (peracetic acid), sodium hypochlorite (bleach, when properly diluted), and quaternary ammonium compounds.

Suitable pesticides include, but are not limited to, copper-based fungicides (e. g., copper sulfate, copper hydroxide), Bacillus thuringiensis (a biological control for various caterpillar pests), Spinosad (derived from the soil bacterium Saccharopolyspora spinosa and effective against a range of pests like caterpillars, thrips, and leafminers), neem oil (derived from the seeds of the neem tree and effective against various pests and fungi), pyrethrin (derived from chrysanthemum flowers, effective against a broad range of insects), insecticidal soaps (potassium salts of fatty acids and effective against soft-bodied pests like aphids and whiteflies, horticultural oils (highly refined petroleum oils or plant-based oils effective against a variety of pests and some diseases), diatomaceous earth (composed of crushed diatom fossils, and effective against crawling insects, biological fungicides (microbial-based products like Bacillus subtilis, Trichoderma spp., and Streptomyces spp. and effective against various fungal diseases.

When methyl decanoate is used for removing the apical meristem, it may be used at a concentration in the range of about 0.01 M and 10 M, of about 0.01 M and 1 M, of about 0.01 M and 0.6 M, or of about 0.1 M and 0.6 M. For the preparation of 0.05 M and 0.06M methyl decanoate, 9.3 g/L and 11.17 g/L, respectively, or 35.15 g/gallon and 42.25 g/gallon, respectively are prepared. One thousand grams of methyl decanoate is mixed with an equal weight of the surfactant polyoxyethylene sorbitan monolaurate. The fatty acid is thoroughly mixed with the surfactant before any water is added. Water is slowly added until the mixture thickens or forms a gel. With continuous mixing, water is added to a volume of 28.45 gallons to form the 0.05 M methyl decanoate: polyoxyethylene sorbitan monolaurate composition. With continuous mixing, water is added to a volume of 23.67 gallons to form the 0.06 M methyl decanoate: polyoxyethylene sorbitan monolaurate composition.

The following examples are provided for illustrative purposes only and not intended to limit the scope of the claims.

### Example 1: Increased Seed Oil Content in Soybean Treated with Methyl Esters of Fatty Acids

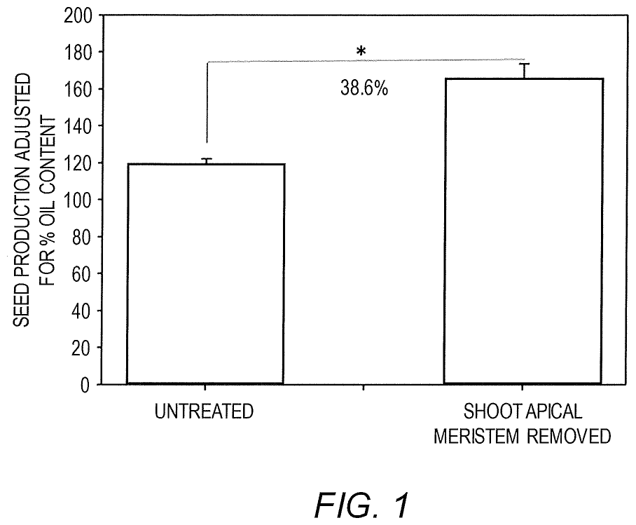

Soybean varieties are planted and when the plants reach the V3 to V4 growth stage, they are sprayed with a formulation containing methyl esters of fatty acids C₆, C₈, C₁₀, and C₁₂. The chemical formulation also contains the surfactant polysorbate 20 (sold under the tradename TWEEN® 20) in equal weight in grams to the total weight in grams of the fatty acid methyl esters. The chemical formulation is applied using a backpack sprayer. The field plots also contain untreated control plants of two soybean varieties. At maturity, the soybean seeds are harvested, weighed, and counted. The oil and protein contents of the seeds are determined. See, e.g., FIG. 1. The number of seeds and oil content produced per hectare of the treated plants is compared to untreated control soybean plants.

### Example 2: Increased Seed Oil Content in Canola Treated with Methyl Esters of Fatty Acids

Two canola varieties are planted. Canola plants are sprayed between growth stage 12 and 13 with the formulation containing methyl esters of fatty acids C₆, C₈, C₁₀, and C₁₂. The chemical formulation also contains the surfactant polysorbate 20 (sold under the tradename TWEEN® 20) in equal weight in grams to the total weight in grams of the fatty acid methyl esters. The chemical formulation is applied using a backpack sprayer. The field plots contain untreated control plants of the two canola varieties. At maturity, canola seeds are harvested, weighed, and counted. The oil content of the seeds is determined. The number of seeds and oil content of the same are compared for the treated and untreated canola plants.

The total quantity of seeds and seed oil content produced per hectare in a 12-month time period from crop 1 and crop 2 of treated and untreated plants is determined and compared.

What is claimed is:

1. A method of increasing seed yield of an oilseed plant produced per hectare in a 12-month time period comprising applying an effective amount of a composition comprising one or more fatty acids and one or more surfactants to the shoot apical meristem of a population of oilseed plants thereby increasing seed yield of the oilseed plant produced per hectare in a 12-month time period as compared to a population of untreated control plants.

2. The method of claim 1, wherein the one or more fatty acids is selected from the group consisting of methyl decanoate, 1-decanol, and methyl nonanoate.

3. The method of claim 1, wherein the one or more fatty acids is one or a combination of methyl esters of C6, C8, C10, and C12 fatty acids.

4. The method of claim 3, wherein composition comprises about 4% of the methyl ester of the C6 fatty acid; about 56% of the methyl ester of the C8 fatty acid; about 38% of the methyl ester of the C10 fatty acid; and about 2% of the methyl ester of the C12 fatty acid, based on total fatty acid of the composition.

5. The method of claim 1, wherein the total amount of the one or more surfactants is equal in weight to the total weight of the one or more fatty acids.

6. The method of claim 1, wherein the one or more surfactants are selected from the group consisting of polyoxyethylene sorbitan esters, polyoxyethylene alcohols, alkylarylpolyether alcohols, phthalic glycerol alkyl resins, ethoxylated imidazoline, decanoic acid, diglycol oleate, polyether sulfonates, alkylaryl sulfonates, and mixtures thereof.

7. The method of claim 1, wherein the effective amount of the composition kills or inhibits the growth of the shoot apical meristem of the population of oilseed plants.

8. The method of claim 1, wherein the population of oilseed plants is selected from the group consisting of soybean, rapeseed, canola, camelina, sorghum, safflower, sunflower, corn, sesame, cotton, cress, peanut, castor, flax, cress, mustard, quinoa, chia, cuphea, and amaranth plants.

9. The method of claim 8, wherein the population of oilseed plants is:
(a) soybean and the composition is applied to the shoot apical meristem of the soybean between
(i) growth stages 1 and 2,
(ii) growth stages 2 and 3,
(iii) growth stages 3 and 4,
(iv) growth stages 4 and 5, or
(v) growth stages 5 and 6;
(b) canola and the composition is applied to the shoot apical meristem of the canola between
(i) growth stages 10 and 11,
(ii) growth stages 11 and 12,
(iii) growth stages 12 and 13,
(iv) growth stages 13 and 14,
(v) growth stages 14 and 15,
(vi) growth stages 15 and 16,
(vii) growth stages 16 and 17,
(viii) growth stages 18 and 19, or
(ix) growth stages 19 and 20;
(c) camelina and the composition is applied to the shoot apical meristem of the camelina between
(i) growth stages 10 and 11,
(ii) growth stages 11 and 12,
(iii) growth stages 12 and 13,
(iv) growth stages 13 and 14,
(v) growth stages 14 and 15,
(vi) growth stages 15 and 16,
(vii) growth stages 16 and 17,
(viii) growth stages 18 and 19, or
(ix) growth stages 19 and 21;
(d) sunflower and the composition is applied to the shoot apical meristem of the sunflower between
(i) growth stages 10 and 12,
(ii) growth stages 12 and 14,
(iii) growth stages 14 and 16,
(iv) growth stages 16 and 18, or
(v) growth stages 19 and 30;
(e) corn and the composition is applied to the shoot apical meristem of the corn between
(i) growth stages 9 and 10,
(ii) growth stages 10 and 11,
(iii) growth stages 11 and 12,
(iv) growth stages 12 and 13,
(v) growth stages 13 and 14,
(vi) growth stages 14 and 15,
(vii) growth stages 15 and 16,
(viii) growth stages 16 and 17,
(ix) growth stages 17 and 18,
(x) growth stages 18 and 19, or
(xi) growth stages 19 and 30;
(f) cotton and the composition is applied to the shoot apical meristem of the cotton between
(i) growth stages 10 and 11,
(ii) growth stages 11 and 12,
(iii) growth stages 12 and 13,
(iv) growth stages 13 and 14,
(v) growth stages 14 and 15,
(vi) growth stages 15 and 16,
(vii) growth stages 16 and 17,
(viii) growth stages 18 and 19, or
(ix) growth stages 19 and 21;
(g) peanut and the composition is applied to the shoot apical meristem of the peanut between
(i) growth stages 10 and 11,
(ii) growth stages 11 and 12,
(iii) growth stages 12 and 13,
(iv) growth stages 13 and 14,
(v) growth stages 14 and 15,
(vi) growth stages 15 and 16,
(vii) growth stages 16 and 17,
(viii) growth stages 18 and 19, or
(ix) growth stages 19 and 21;
(h) sorghum and the composition is applied to the shoot apical meristem of the sorghum between
(i) growth stages 0 and 1,
(ii) growth stages 1 and 2,
(iii) growth stages 2 and 3,
(iv) growth stages 3 and 4,
(v) growth stages 4 and 5, or
(vi) growth stages 5 and 6.

10. A method of increasing a quantity of seed oil produced per hectare in a 12-month time period comprising applying an effective amount of a composition comprising one or more fatty acids and one or more surfactants to the shoot apical meristem of a population of oilseed plants thereby increasing the quantity of seed oil produced per hectare in a 12-month time period by the population of oilseed plants as compared to a population of untreated control plants.

11. The method of claim 10, wherein the one or more fatty acids is selected from the group consisting of methyl decanoate, 1-decanol, and methyl nonanoate.

12. The method of claim 10, wherein the one or more fatty acids is one or a combination of methyl esters of C6, C8, C10, and C12 fatty acids.

13. The method of claim 12, wherein composition comprises about 4% of the methyl ester of the C6 fatty acid; about 56% of the methyl ester of the C8 fatty acid; about 38% of the methyl ester of the C10 fatty acid; and about 2% of the methyl ester of the C12 fatty acid, based on total fatty acid of the composition.

14. The method of claim 10, wherein the total amount of the one or more surfactants is equal in weight to the total weight of the one or more fatty acids.

15. The method of claim 10, wherein the one or more surfactants are selected from the group consisting of polyoxyethylene sorbitan esters, polyoxyethylene alcohols, alkylarylpolyether alcohols, phthalic glycerol alkyl resins, ethoxylated imidazoline, decanoic acid, diglycol oleate, polyether sulfonates, alkylaryl sulfonates, and mixtures thereof.

16. The method of claim 10, wherein the effective amount of the composition kills or inhibits the growth of the shoot apical meristem of the population of oilseed plants.

17. The method of claim 10, wherein the effective amount increases seed oil content, seed protein content, and/or seed yield per hectare compared to a population of untreated control plants.

18. The method of claim 10, wherein the population of oilseed plants is selected from the group consisting of soybean, rapeseed, canola, camelina, sorghum, safflower, sunflower, corn, sesame, cotton, cress, peanut, castor, flax, cress, mustard, quinoa, chia, cuphea, and amaranth plants.

19. The method of claim 18, wherein the population of oilseed plants is:
(a) soybean and the composition is applied to the shoot apical meristem of the soybean between
(i) growth stages 1 and 2,
(ii) growth stages 2 and 3,
(iii) growth stages 3 and 4,
(iv) growth stages 4 and 5, or
(v) growth stages 5 and 6;
(b) canola and the composition is applied to the shoot apical meristem of the canola between
(i) growth stages 10 and 11,
(ii) growth stages 11 and 12,
(iii) growth stages 12 and 13,
(iv) growth stages 13 and 14,
(v) growth stages 14 and 15,
(vi) growth stages 15 and 16,
(vii) growth stages 16 and 17,
(viii) growth stages 18 and 19, or
(ix) growth stages 19 and 20;
(c) camelina and the composition is applied to the shoot apical meristem of the camelina between
(i) growth stages 10 and 11,
(ii) growth stages 11 and 12,
(iii) growth stages 12 and 13,
(iv) growth stages 13 and 14,
(v) growth stages 14 and 15,
(vi) growth stages 15 and 16,
(vii) growth stages 16 and 17,
(viii) growth stages 18 and 19, or
(ix) growth stages 19 and 21;
(d) sunflower and the composition is applied to the shoot apical meristem of the sunflower between
(i) growth stages 10 and 12,
(ii) growth stages 12 and 14,
(iii) growth stages 14 and 16,
(iv) growth stages 16 and 18, or
(v) growth stages 19 and 30;
(e) corn and the composition is applied to the shoot apical meristem of the corn between
(i) growth stages 9 and 10,
(ii) growth stages 10 and 11,
(iii) growth stages 11 and 12,
(iv) growth stages 12 and 13,
(v) growth stages 13 and 14,
(vi) growth stages 14 and 15,
(vii) growth stages 15 and 16,
(viii) growth stages 16 and 17,
(ix) growth stages 17 and 18,
(x) growth stages 18 and 19, or
(xi) growth stages 19 and 30;
(f) cotton and the composition is applied to the shoot apical meristem of the cotton between
(i) growth stages 10 and 11,
(ii) growth stages 11 and 12,
(iii) growth stages 12 and 13,
(iv) growth stages 13 and 14,
(v) growth stages 14 and 15,
(vi) growth stages 15 and 16,
(vii) growth stages 16 and 17,
(viii) growth stages 18 and 19, or
(ix) growth stages 19 and 21;
(g) peanut and the composition is applied to the shoot apical meristem of the peanut between
(i) growth stages 10 and 11,
(ii) growth stages 11 and 12,
(iii) growth stages 12 and 13,
(iv) growth stages 13 and 14,
(v) growth stages 14 and 15,
(vi) growth stages 15 and 16,
(vii) growth stages 16 and 17,
(viii) growth stages 18 and 19, or
(ix) growth stages 19 and 21;
(h) sorghum and the composition is applied to the shoot apical meristem of the sorghum between
(i) growth stages 0 and 1,
(ii) growth stages 1 and 2,
(iii) growth stages 2 and 3,
(iv) growth stages 3 and 4,
(v) growth stages 4 and 5, or
(vi) growth stages 5 and 6.
